(19) United States
(12) Patent Application Publication
(10) Pub. No.: US 2003/0109504 A1
(43) Pub. Date: Jun. 12, 2003

(54) Treatment of movement disorders with metabotropic glutamate receptors antagonist

(76) Inventors: Jonathan Brotchie, Manchester (GB); Michael Hill, Oldham (GB); Alan Crossman, Manchester (GB)

Correspondence Address: Nixon & Vanderhye, 8th Floor, 1100 North Glebe Road, Arlington, VA, 22201-4714, US

(21) Appl. No.: 10/239,710

(22) PCT Filed: Mar. 23, 2001
(86) PCT No.: PCT/GB01/01279

(30) Foreign Application Priority Data
Mar. 25, 2000 (GB) 0007193.6

(51) Int. Cl. A61K 31/655; A61K 31/44

(52) U.S. Cl. 514/150; 514/277

(57) ABSTRACT

The present invention relates to the use of compounds, which inhibit metabotropic glutamate receptor activity, or activation, for use in the treatment of movement disorders associated with a poverty of movement (e.g. Parkinson's disease). The compounds are particularly useful when used in combination with another therapeutic agent and surprisingly reduce the extent and incidence of side effects (e.g. dyskinesia) associated with such therapeutic agents.

The present invention relates to the treatment of movement disorders associated with a poverty of movement and more particularly to the treatment of parkinsonism.

Movement and other disorders due to dysfunction of the basal ganglia and related brain structures are of major socio-economic importance. Such disorders can occur as a consequence of inherited or acquired disease, idiopathic neurodegeneration or they may be iatrogenic. The spectrum of disorders is very diverse, ranging from those associated with poverty of movement (akinesia, hypokinesia, bradykinesia) and hypertonia (e.g. Parkinson's disease, some forms of dystonia) to the involuntary movement disorders (hyperkinesias or dyskinesias e.g. Huntington's disease, levodopa-induced dyskinesia, ballism, some forms of dystonia).

Parkinson's disease and related conditions represent one of the most prevalent diseases associated with poverty of movement. Parkinsonian symptoms manifest as a syndrome of symptoms characterised by slowness of movement (bradykinesia), rigidity and/or tremor. Parkinsonian symptoms are seen in a variety of conditions, most commonly in idiopathic parkinsonism (i.e. Parkinson's Disease) but also following treatment of schizophrenia, manganese poisoning and head injury.

It is now widely appreciated that the primary pathology underlying Parkinson's disease is degeneration, in the brain, of the dopaminergic projection from the substantia nigra to the striatum. This realisation has led to the widespread use of dopamine-replacing agents (e.g. L-DOPA and apomorphine) as symptomatic treatments for Parkinson's disease and such treatments have undoubtedly been successful in increasing the quality of life of patients suffering from Parkinson's disease.

However, dopamine-replacement treatments do have limitations, especially following long-term treatment. Problems can include a wearing-off of the anti-parkinsonian efficacy of the treatment and in particular the appearance of a range of side effects. These side effects may manifest as dyskinesias such as chorea and dystonia. Dyskinesia can be seen either when the patient is undergoing dopamine-replacement therapy (in the case of chorea and/or dystonia) or even when off therapy (when dystonia is prevalent). Ultimately, these side-effects severely limit the usefulness of dopaminergic treatments.

Many attempts have been made to develop novel dopamine replacement therapies which will obviate or mitigate these side effects. However such attempts have generally met with limited success and there remains a need to develop new and improved ways in which Parkinsonism may be treated.

Other movement disorders associated with poverty of movement are even more difficult to treat than Parkinson's disease and some lack any effective therapy. Such conditions include Wilson's disease, progressive supranuclear palsy, some forms of dystonia and drug/toxin-induced parkinsonism.

According to a first aspect of the present invention, there is provided a use of a compound which inhibits metabotropic glutamate receptor activity, or activation, for the manufacture of a medicament for the treatment of movement disorders associated with a poverty of movement.

According to a second aspect of the present invention, there is provided a method for the treatment of movement disorders associated with a poverty of movement comprising administering to a person or animal in need of such treatment a therapeutically effective amount of a compound which inhibits metabotropic glutamate receptor activity.

By “movement disorder associated with a poverty of movement” we mean a medical condition characterised by akinesia, hypokinesia or bradykinesia and also conditions characterised by hypertonia. Such disorders include Wilson's disease, progressive supranuclear palsy, some forms of dystonia and in particular parkinsonism.

Metabotropic glutamate receptors are a subclass of glutamate receptors which are found in neural tissues. These receptors can be further divided into Group I, II and III metabotropic glutamate receptors based upon their pharmacology and may be further subdivided into the receptor types described in Table 1.

TABLE 1
Types of metabotropic Glutamate Receptor (mGluR)

| mGluR receptors | Group I | Group II | Group III |
|---|---|---|---|
| | mGlu₁ | mGlu₂ | MGlu₄ (4a, 4b) |
| | (1a, 1b, 1c, 1d) | mGlu₃ | MGlu₆ |
| | mGlu₅ | | MGlu₇ (7a, 7b) |
| | (5a, 5b) | | MGlu₈ (8a, 8b, 8c) |

We have found that compounds which inhibit metabotropic glutamate receptor activity are useful in the treatment of movement disorders as defined herein.

The inventors have established that compounds used according to the present invention are associated with less side effects than most conventional therapies. For instance, side effects such as dyskinesias (e.g. chorea and dystonia) do not develop, or develop to a lesser extent, when compounds that inhibit metabotropic glutamate receptors are used. Furthermore when the compounds are used in combination therapy, we have found that either (i) less of the conventional agent is required (which leads to a reduction in the side effects associated with conventional therapies); or (ii) the compound that inhibits metabotropic glutamate receptor activity acts to reduce side effects, such as dyskinesia, associated with the known therapies.

By “dyskinesia” we mean the development in a subject of abnormal involuntary movements. These movements may manifest as chorea (irregular, involuntary movements of the body, especially the face and extremities) or dystonia (disorder or lack of muscle tonicity). Such movements include ballistic movements and athetoid movements of the trunk, limbs and facial musculature.

The invention is based upon our studies relating to the neural mechanisms underlying movement disorders. Although we do not wish to be bound by any hypothesis, we believe that movement disorders involve abnormal activity of basal ganglia output pathways and in many cases this is brought about by abnormal function of striatal efferent pathways. These consist of a “direct” pathway to the medial or internal segment of the globus pallidus and the pars reticulata of the substantia nigra and an “indirect” pathway to the lateral or external segment of the globus pallidus. One of the pathophysiological hallmarks of parkisonism is over activity of the indirect striatal output pathway (this appears to be caused by underactivity at dopamine D₂-receptors). Dopamine replacement therapy reverses this. However the limitations of such Dopamine replacement therapy results from stimulation of the “direct” pathway via D₁ receptors. We believe that compounds which inhibit metabotropic glutamate receptor activity, or activation, reduce the effect of dopamine on the direct striatal output pathway and thereby relieve the symptoms of movement disorders such as parkinsonism and other hypokinetic disorders in a more effective way.

Several classes of compound may be used according to the invention to inhibit metabotropic glutamate receptor activity. These compounds include:

(i) compounds which attenuate transmission at metabotropic glutamate receptors (e.g. metabotropic glutamate receptor antagonists and partial agonists; anti-sense molecules for the metabotropic glutamate receptor gene; and molecules which attenuate metabotropic glutamate receptor-effector coupling);

(ii) compounds which inverse stimulate metabotropic glutamate receptors (i.e. inverse agonists);

(iii) compounds which inhibit synthesis of endogenous metabotropic glutamate receptor agonists by decreasing the synthesis of precursors or decreasing the conversion of precursors into metabotropic glutamate receptor-activating ligands;

(iv) compounds which inhibit release of metabotropic glutamate receptor agonists(e.g. Enadoline, WIN55-212,2, lamotrigine, IL-1β, clonidine, sodium nitroprusside, N6-cyclopentyladenosine, imetit, riluzole);

(v) compounds which increase the rate of inactivation or metabolism of metabotropic glutamate receptor agonists (e.g. glutamine synthetase, glutamic acid decarboxylase); and

(vi) compounds which reduce metabotropic glutamate receptor expression and/or transcription.

The compound may modulate any type of glutamate receptor provide that metabotropic glutamate receptor activity is inhibited. However it is preferred that the compound selectively inhibits the activity of metabotropic glutamate receptors. By “selectively” we mean the compound inhibits metabotropic glutamate receptor activity or activation to a greater extent or at lower doses than other types of glutamate receptor.

It is more preferred that compounds which inhibit the activity of type I metabotropic glutamate receptors are used. This is because we have found that compounds that specifically modulate type I metabotropic glutamate receptor activity have most efficacy for treating hypokinetic movement disorders. However it should be appreciated that compounds that inhibit type II or III receptor activity are useful for treating movement disorders and may therefore be used according to the invention.

Metabotropic glutamate receptor antagonists ((i) above) are preferred compounds for use according to the invention. Examples of selective antagonists, which are suitable for treating movement disorders, are listed in table 2. The group I antagonists listed in Table 2 are preferred antagonists.

TABLE 2
Selective Ligands for metabotropic glutamate receptors

| | |
|---|---|
| Group I | (S)-4-Carboxy-3-hydroxyphenylglycine; |
| antagonists | 7-(Hydroxyimino)cyclopropa[β]chromen-1α- |
| | carboxylate ethyl ester (RS)-1-Aminoindan- |
| | 1,5-dicarboxylic acid (AIDA); |
| | 2-Methyl-6-(phenylethynyl)pyridine (MPEP); |
| | 2-Methyl-6-(2-phenylethenyl)pyridine (SIB-1893); |
| | 6-Methyl-2-(phenylazo)-3-pyridinol (SIB-1757); |
| | (S)-(+)-α-Amino-4-carboxy-2-methylbenzeneacetic acid; |
| Group II | (2S,3S,4S)-2-Methyl-2-(carboxycyclopropyl)glycine; |
| antagonists | (2S-2-amino-2-(1S,2S-2-carboxycycloprop-1-yl)-3- |
| | (xanth-9-yl)propanoic acid); |
| | (2S)-α-Ethylglutamic acid (EGLU); |
| Group III | (S)-2-Amino-2-methyl-4-phosphonobutanoic acid; |
| antagonists | (RS)-α-Cyclopropyl-4-phosphonophenylglycine; |
| | (RS)-α-Methylserine-O-phosphate (MSOP); |

It will be appreciated from Table 1 (above) that Group I mGluR include mGlu₁ (1a, 1b, 1c, 1d etc) and mGlu₅ (5a, 5b etc) receptors. We have found that compounds that inhibit mGlu₅ receptors are particularly useful for treating movement disorders. For instance, 2-methyl-6-(phenylethynyl)-pyridine (MPEP), SIB-1757 or SIB-1893 are particularly useful in this respect.

The compounds (and compositions or medicaments containing them) may be used to treat many types of movement disorder associated with a poverty of movement (i.e. akinesia, hypokinesia or bradykinesia). For instance the compounds may be used to treat Wilson's disease, progressive supranuclear palsy, some forms of dystonia and in particular parkinsonism (e.g. idiopathic Parkinson's disease, post-encephalitic parkinsonism, parkinsonism resulting from head injury, toxin induced parkinsonism).

The compounds are particularly useful when combined with another anti-parkinsonism therapy. For instance compounds which metabotropic glutamate receptor antagonists may be combined with known anti-parkinsonian therapies (e.g. anti-parkinsonian agents such as L-DOPA or apomorphine) to significantly alleviate the parkinsonian symptoms. In fact certain combinations of compounds which inhibit metabotropic glutamate receptor activity and anti-parkinsonian therapies/agents actually resulted in a synergistic effect. Furthermore the inventors have found that every contemporary anti-parkinsonian therapy they have investigated is surprisingly more effective when combined with treatment with compounds that inhibit metabotropic glutamate receptor activity or activation. Therefore the inventors expect the compounds may be beneficially used with any known anti-parkinsonian therapy and also with therapies not yet contemplated.

Accordingly a preferred embodiment of the invention involves the use of compounds that inhibit metabotropic glutamate receptor activity or activation in combination with another therapeutic agent used for treating movement disorders associated with a poverty of movement.

The compounds are preferably combined according to the invention with anti-parkisonism therapies that utilise specific therapeutically active agents. Preferred agents include Chloro-APB, L-DOPA, apomorphine, ropinirole, pramipexole, cabergoline, bromcriptine, lisuride, quinpirole and pergolide. The compounds may also be used in combination with agents such as other dopamine D₁-receptor agonists, other dopamine. D₂-receptor agonists, other mixed dopamine receptor agonists, adenosine A_{2A}-receptor antagonists, muscarinic M₄-antagonists, nicotinic agonists, delta opioid agonists and NMDA receptor antagonists.

The compounds may also be used in combination with other therapies for reducing the activity of basal ganglia outputs. For instance cell implantation/ transplantation, gene delivery systems (see below), subthalamic nucleus lesions/deep brain stimulation, and Gpi lesions/deep brain stimulation.

A surprising advantage (illustrated in Example 2) of the abovementioned combination therapies is that the compounds that inhibit metabotropic glutamate receptor activity have the effect of reducing the extent and incidence of side effects (e.g. dyskinesia) associated with known therapeutic agents. Accordingly the combination therapy represents a significant improvement over conventional monotherapies with agents such as L-DOPA because there is a significant reduction in side effects such as dyskinesia.

The compounds used according to the invention may be used to treat existing movement disorders but may also be used when prophylactic treatment is considered medically necessary. For instance, following a head injury when it is feared parkinsonian symptoms may develop.

The compounds a preferably used to treat human subjects suffering from Parkinson's disease.

The compound that inhibits the activity of metabotropic glutamate receptors may be formulated in a number of ways depending, in particular on the manner in which the composition is to be used. Thus, for example, the compound may be formulated in the form of a powder, tablet, capsule, liquid, ointment, cream, gel, hydrogel, aerosol, spray, micelle, liposome or any other suitable form that may be administered to a person or animal. It will be appreciated that the vehicle for the compound should be one that is well tolerated by the subject to whom it is given and enables delivery of the compound to the brain.

When used in a combination therapy, the compounds may be formulated in a single composition which also comprises another anti-parkinsonism agent. Alternatively the compound and agent may be formulated in separate formulations and co-administered to the subject either simultaneously or sequentially.

The compounds may be used in a number of ways. For instance, systemic administration may be required in which case the compound may be contained within a composition which may, for example, be ingested orally in the form of a tablet, capsule or liquid. Alternatively the compound may be administered by injection into the blood stream. Injections may be intravenous (bolus or infusion) or subcutaneous (bolus or infusion). The compounds may also be administered by inhalation or intranasally.

Compounds inhibiting metabotropic glutamate receptor activity may also be administered centrally by means of intracerebral, intracerebroventricular, or intrathecal delivery.

The compound may also be incorporated within a slow or delayed release device. Such devices may, for example, be inserted under the ski and the compound may be released over weeks or even months. On the other hand, transdermal delivery might be used to achieve the same end. Such devices may be particularly useful for patients requiring long term and/or continuous therapy for Parkinson's disease. The devices may be particularly advantageous when a compound is used which would normally require frequent administration (e.g. at least daily ingestion of a tablet or daily injection).

It will be appreciated that the amount of a compound required is determined by biological activity and bioavailability which in turn depends on the mode of administration, the physicochemical properties of the compound employed and whether or not the compound is to be used in combination therapy.

The frequency of administration will also be influenced by the above mentioned factors and particularly the half-life of the compound within the subject being treated.

Known procedures, such as those conventionally employed by the pharmaceutical industry (e.g. in vivo experimentation, clinical trials etc), may be used to establish specific formulations of compounds (whether formulated with the agent or otherwise) and precise therapeutic regimes (such as daily doses of the compounds and the frequency of administration).

Generally, a daily dose of between 0.01 μg/kg of body weight and 1.0 g/kg of body weight of a compound which inhibits metabotropic glutamate receptor activity may be used for the treatment of the movement disorders depending upon which specific compound is used. More preferably the daily dose is between 0.01 mg/kg of body weight and 100 mg/kg of body weight and most preferably 0.05-10 mg/kg of body weight.

Purely by way of example a suitable dose of AIDA for use in conjunction with chloro-APB in subjects with Parkinson's disease is between 0.1 mgs/kg/day and 100 mgs/kg/day (depending upon the health status of the individual). It is preferred that between 0.1 mgs/kg/day and 50 mgs/kg/day of ADA is given to a person daily and most preferred that about 5 mgs/kg/day are given.

By way of further example a suitable dose of SIB-1893 for use in conjunction with L-DOPA in subjects with Parkinson's disease is between 0.1 mgs/kg/day and 100 mgs/kg/day (depending upon the health status of the individual). It is preferred that between 0.1 mgs/kg/day and 50 mgs/kg/day of SIB-1893 is given to a person daily and most preferred that about 20 mgs/kg/day are given.

Daily doses may be given as a single administration (e.g. a daily tablet for oral consumption or as a single daily injection). Alternatively the compound used may require administration twice or more times during a day. As an example, 1-Aminoindan-1,5-dicarboxylic acid (AIDA) may be administered as two (or more depending upon the severity of the, condition) daily doses of between 25 mgs and 5000 mgs in tablet form. Alternatively a slow release device may be used to provide optimal doses to a patient without the need to administer repeated doses.

A preferred means of using protein or peptide compounds which inhibit metabotropic glutamate receptor activity for the treatment of a disorder characterised by a poverty of movement is to deliver the compound to the brain by means of gene therapy. For instance, gene therapy may be used to decrease expression of metabotropic glutamate receptors, increase expression of enzyme(s) responsible for the degradation of endogenous metabotropic glutamate receptor agonists (e.g.enzymes which metabolise glutamate per se), increase expression of a protein which promotes breakdown or desensitisation of metabotropic glutamate receptors or increase expression of a protein which promotes breakdown of metabotropic glutamate receptor agonists. Therefore according to a fourth aspect of the present invention there is provided a delivery system for use in a gene therapy technique, said delivery system comprising a DNA molecule encoding for a protein which directly or indirectly inhibits metabotropic glutamate receptor activity, said DNA molecule being capable of being transcribed to allow the expression of said protein and thereby treat a movement disorder associated with poverty of movement.

The delivery systems according to the fourth aspect of the invention are highly suitable for achieving sustained levels of a protein which directly or indirectly inhibits metabotropic glutamate receptor activity over a longer period of time than is possible for most conventional therapeutic regimes. The delivery system may be used to induce continuous protein expression from cells in the brain that have been transformed with the DNA molecule. Therefore, even if the protein has a very short half-life as an agent in vivo, therapeutically effective amounts of the protein may be continuously expressed from the treated tissue.

Furthermore, the delivery system of the invention may be used to provide the DNA molecule (and thereby the protein which is an active therapeutic agent) without the need to use conventional pharmaceutical vehicles such as those required in tablets, capsules or liquids.

The delivery system of the present invention is such that the DNA molecule is capable of being expressed (when the delivery system is administered to a patient) to produce a protein which directly or indirectly has activity for inhibiting metabotropic glutamate receptor activity. By “directly” we mean that the product of gene expression per se has the required activity. By “indirectly” we mean that the product of gene expression undergoes or mediates (e.g. as an enzyme) at least one further reaction to provide a compound effective for inhibiting metabotropic glutamate receptor activity and thereby treating the movement disorder.

The DNA molecule may be contained within a suitable vector to form a recombinant vector. The vector may for example be a plasmid, cosmid or phage. Such recombinant vectors are highly useful in the delivery systems of the invention for transforming cells with the DNA molecule.

Recombinant vectors may also include other functional elements. For instance, recombinant vectors can be designed such that the vector will autonomously replicate in the cell. In this case, elements which induce DNA replication may be required in the recombinant vector. Alternatively the recombinant vector may be designed such that the vector and recombinant DNA molecule integrates into the genome of a cell. In this case DNA sequences which favour targeted integration (e.g. by homologous recombination) are desirable. Recombinant vectors may also have DNA coding for genes that may be used as selectable markers in the cloning process.

The recombinant vector may also further comprise a promoter or regulator to control expression of the gene as required.

The DNA molecule may (but not necessarily) be one that becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecule will stop when the transformed cells die or stop expressing the protein (ideally when the movement disorder has been treated or prevented).

The delivery system may provide the DNA molecule to the subject without it being incorporated in a vector. For instance, the DNA molecule may be incorporated within a liposome or virus particle. Alternatively the “naked” DNA molecule may be inserted into a subject's cells by a suitable means e.g. direct endocytotic uptake.

The DNA molecule may be transferred to the cells of a subject to be treated by transfection, infection, microinjection, cell fusion, protoplast fusion or ballistic bombardment. For example, transfer may be by ballistic transfection with coated gold particles, liposomes containing the DNA molecule, viral vectors (e.g. adenovirus) and means of providing direct DNA uptake (e.g. endocytosis) by application of the DNA molecule directly to the brain topically or by injection.

The delivery system may also comprise a further DNA molecule (which may optionally be incorporated within the same vector) which encodes for an anti-parkinsonian agent. Thus the combination therapy described above may be effected by gene therapy.

An embodiment of the present invention will now be described, by way of example, with reference to the accompanying drawing, in which:

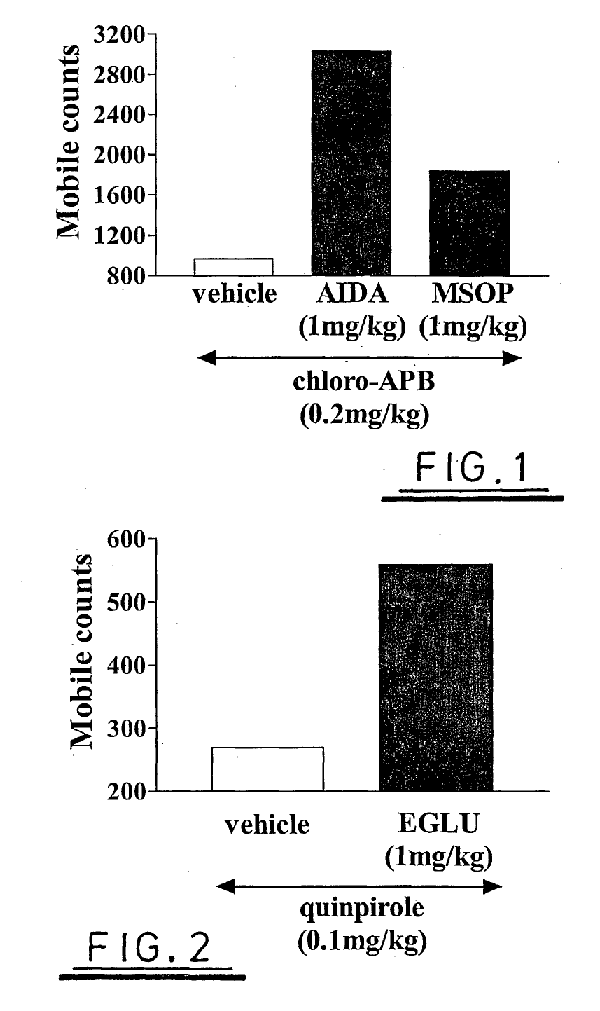

FIG. 1 is a bar chart illustrating the effect of Group I and III metabotropic glutamate receptor antagonists on locomotion following chloro-APB treatment of parkinsonian rats in Example 1;

FIG. 2 is a bar chart illustrating the effect of a Group II metabotropic glutamate receptor antagonist on locomotion following quinpirole treatment of parkinsonian rats in Example 1.

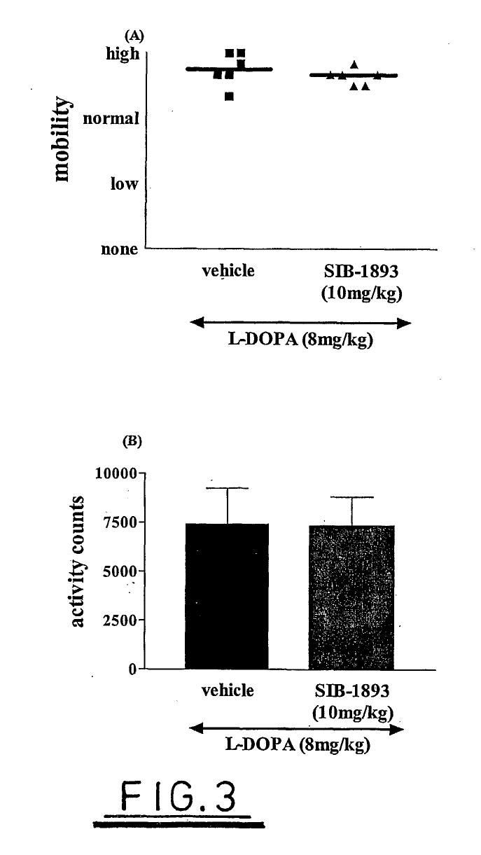

FIG. 3 illustrates the effect of SIB-1893,on L-DOPA-induced (A) mobility and (B) locomotor activity at peak anti-parkinsonian effect in Example 2; individual animal data with the corresponding median is shown in (A) and the mean±s.e mean of total activity counts from 0-120 minutes following drug administration is shown in (B); and

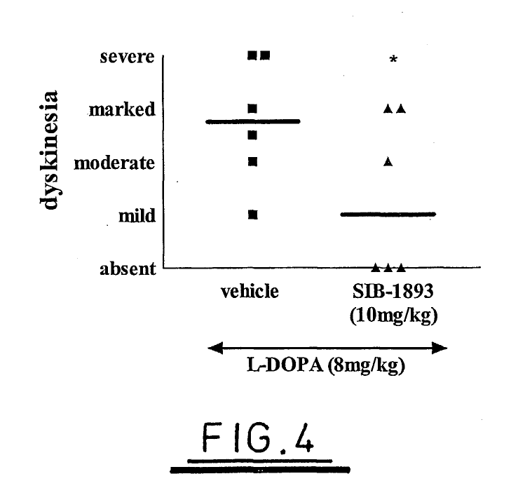

FIG. 4 illustrates the effect of SIB-1893, on L-DOPA-induced dyskinesia at peak anti-parkinsonian effect in Example 2, individual animal data with the corresponding median is shown in the figures; * p<0.05 compared to vehicle+L-DOPA; non-parametric Wilcoxan matched pairs test.

### EXAMPLE 1

The effect of metabotropic glutamate receptor antagonists on the anti-parkinsonian effects of chloro-APB (0.2 mg/kg) or quinpirole (0.1 mg/kg) was assessed in a reserpine-treated rat model of Parkinson's disease.

### 1.1. Methods

1.1.1 Treatments.

Male Sprague-Dawley rats were split into two groups A and B. Rats in both groups were rendered parkinsonian by subcutaneous administration of reserpine (3 mg/kg) for 18 hours.

After the 18 hours Group A were treated with either chloro-APB (0.2 mg/kg) or quinpirole (0.1 mg/kg) and then subdivided into groups A1, A2 and A3. These subgroups were additionally administered the following selective metabotropic glutamate receptor antagonists (1 mg/kg):

A1: (RS)-1-Aminoindan-1,5-dicarboxylic acid (AIDA—Group I metabotropic glutamate receptor antagonist);

A2: (2S)-alpha-Ethylglutamic acid (EGLU—Group II metabotropic glutamate receptor antagonist); and

A3: (RS)-alpha-Methylserine-O-phosphate (MSOP—Group III metabotropic glutamate receptor antagonist).

Group B were treated with chloro-APB (0.2 mg/kg) and vehicle for the antagonists only.

1.1.2 Assessment of Activity and Mobility.

The locomotion of the rats in Groups A and B was measured over a one hour period using Benwick locomotor monitors. These locomotion monitors consist of a visually-shielded open-field arena, the perimeter of which is surrounded by a series of infra-red beams arranged at 5 cm intervals. PC-based software (Amlogger) assesses the number of beams broken. The number of beams broken as part of a locomotor movement (mobile counts) or the number of beam breaks while the animal is not locomoting (static counts) were measured. In addition, the system assesses the time for which animals are mobile or static.

### 1.2 Results

FIG. 1 illustrates that total mobile counts for AIDA (a group I antagonist) and chloro-APB; and MSOP (a group m antagonist) and chloro-APB treated animals was greater than those treated with vehicle and chloro-APB only.

FIG. 2 illustrates that EGLU (a group II antagonist) and quinpirole treated animals also had greater mobility than those treated with vehicle and quinpirole only.

### 1.3. Conclusion

These data illustrate that total mobile counts for animals on a combination therapy was significantly greater than those treated with known anti-parkinsonian agents (Chloro-APB or quinpirole) and vehicle only. This demonstrates that mobility is improved and therefore the Parkinsonian state is improved in animals given the combination therapy according to the present invention. The inventors believe this occurs because the compounds increase D₁-dopamine receptor-dependent locomotion (i.e. increased locomotor activity via the direct striatal output pathway).

These data further illustrate that Group I, II and III antagonists are each effective according to the present invention whereas Group I antagonists (e.g. AIDA), which are preferred compounds for use according to the invention, are particularly effective.

### EXAMPLE 2

The effect of the mGluR group I (selective for mGlu₅) receptor antagonist SIB-1893 in combination with L-DOPA was assessed in the MPTP-lesioned marmoset model of Parkinson's disease. The ability of the traditional anti-parkinsonian agent L-DOPA to alleviate symptoms was compared with the combined therapy.

### 2.1. Methods

2.1.1 Preparation of MPTP-Lesioned Marmoset Model of Parkinson's Disease

Marmosets (Callithrix jacchus) (bred in a closed colony at the University of Manchester) are rendered parkinsonian by subcutaneous injection of 2 mg kg⁻¹ MPTP for 5 consecutive days. The marmosets are allowed to recover for a minimum of 10 weeks until their parkinsonism becomes stable. The degree of activity and disability before and after MPTP treatment is assessed using a combination of scales as described below. Animals are then treated with L-DOPA for at least 3 weeks to prime them to elicit dyskinesia.

2.1.2 Assessment of Behaviour

Behaviour was assessed using the following scales:

1) Activity—a quantitative assessment using computer-based activity monitors was obtained every 5 minutes for the duration of the experiment. p1 2) Parkinsonian disability—non-parametric measures based on the following scales:

+P2

Mobility score: 0=no movement, 1=movement of head on the floor of the cage, 2=movement of limbs, but no locomotion, on the floor of the cage, 3=movement of head or trunk on wall of cage or perch, 4=movement of limbs, but no locomotion, on wall of cage or perch, 5=walking around floor of cage or eating from hopper on floor, 6=hopping on floor of cage, 7=climbing onto wall of cage or perch, 8=climbing up and down the walls of the cage or along perch, 9=running, jumping, climbing between cage walls/perch/roof, uses limbs through a wide range of motion and activity.

3) Dyskinesia—non-parametric measures based on the following scale:

Dyskinesia score: 0Absent, 1=Mild, fleeting, 2=Moderate, not interfering with normal activity, 3=Marked, at times interfering with normal activity, 4=Severe, continuous, replacing normal activity.

The behavioural tests were assessed every 30 minutes for 4 hours, by post hoc analysis of video-recordings by an observer blinded to the treatment.

2.1.3 Treatments

Six marmosets received L-DOPA plus vehicle and L-DOPA (8 mg/kg) plus SIB-1893 (10 mg/kg) as shown in FIGS. 1 and 2. The treatments were randomised such that on each day all six marmosets received one of the treatments. There was at least 48 hours washout between treatments.

### 2.2. Results

FIG. 3 illustrates the effect of SIB-1893 treatment on L-DOPA-induced (A) mobility and (B).locomotor activity in the MPTP-lesioned marmoset model of Parkinson's disease. These data demonstrate that the utilisation of SIB-1893 and L-DOPA was as effective as L-DOPA alone in reversing parkinsonism and allowed normal mobility and activity.

FIG. 4 illustrates the effect of SIB-1893 treatment on L-DOPA-induced dyskinesia in the MPTP-lesioned marmoset model of Parkinson's disease. These data demonstrate that SIB-1893 in combination with L-DOPA elicited significantly less severe L-DOPA-induced dyskinesia.

### 2.3. Conclusion

The MPTP-lesioned primate is the ‘gold standard’ preclinical model of Parkinson's disease. In the data presented a good anti-parkinsonian action of the combination (SIB-1893+L-DOPA) and traditional therapy (L-DOPA alone) is seen. However, the combination therapy had an advantage over this traditional therapy. Not only was a reversal of the parkinsonian symptoms of hypokinesia seen but this was also surprisingly accompanied by less involuntary movements, such as dyskinesia. Therefore, the data presented in this Example demonstrate a beneficial therapeutic effect is seen when a combination therapy (L-DOPA+a group I mGluR antagonist) is used to treat Parkinson's disease patients according to a preferred embodiment of the invention.

It will be appreciated that compounds according to the present invention will be just as useful for treating other types of movement disorders associated with a paucity of movement and may also be used as a monotherapy.

1. The use of a selective antagonist of the mGlu₅ receptor for the manufacture of a medicament for the treatment of movement disorders associated with a poverty of movement.

2. The use according to claim 1, wherein the antagonist is selected from the group consisting of:
2-Methyl-6-(phenylethynyl)pyridine,
2-Methyl-6-(2-phenylethenyl)pyridine; and
6-Methyl-2-(phenylazo)-3-pyridinol.

3. The use according to claim 1 or 2, for the treatment of parkinsonism.

4. The use according to claim 3 wherein the parkinsonism is idiopathic Parkinson's disease or post-encephalitic parkinsonism.

5. The use according to claim 3 wherein the parkinsonism results from head injury, the treatment of schizophrenia, drug intoxication or manganese poisoning.

6. The use according to claim 1 or 2 for the treatment of Wilson's disease, progressive supranuclear palsy and dystonia.

7. The use according to any preceding claims wherein the compound is administered in conjunction with an anti-parkinsonian therapy.

8. The use according to claim 7 wherein the anti-parkinsonian therapy is one of cell implantation/transplantation, gene therapy, subthalamic nucleus lesions/deep brain stimulation and Gpi lesions/deep brain stimulation.

9. The use according to claim 7 wherein the anti-parkinsonian therapy comprises administration of an anti-parkinsonian agent.

10. The use according to claim 9 wherein the agent is one of Chloro-APB, L-DOPA, apomorphine, ropinirole, pramipexole, cabergoline, bromcriptine, quinpirole, lisuride, pergolide, a dopamine D₁-receptor agonist, a dopamine D₂-receptor agonist, a mixed dopamine receptor agonist, an adenosine A_{2A}-receptor antagonist, a muscarinic M₄-antagonist, a nicotinic agonist, a delta opioid agonist or a NMDA receptor antagonist.

11. The use according to any proceeding claim for prophylactic treatment.
